(19) United States
(12) Patent Application Publication
(10) Pub. No.: US 2005/0049675 A1
(43) Pub. Date: Mar. 3, 2005

(54) Medical devices and related methods

(75) Inventor: Michael J. Wallace, Bellaire, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System

(21) Appl. No.: 10/811,775

(22) Filed: Mar. 29, 2004

Related U.S. Application Data
(60) Provisional application No. 60/458,813, filed on Mar. 28, 2003.

(51) Int. Cl. A61F 2/06

(52) U.S. Cl. 623/1.13

(57) ABSTRACT

Medical devices for insertion and delivery into a variety of anatomical structures, including vascular structures such as the superior vena cava and the inferior vena cava. In certain embodiments, the medical device includes a hollow body having a main portion and two ends, each end including atraumatic arms; and a graft material is attached to the body; where the body is expandable from a constrained position to an unconstrained position, the atraumatic arms of at least one end are outwardly oriented at an acute angle with respect to the main portion when the body is in the unconstrained position, and graft material contacts one arm that is oriented at an acute angle. Methods for creating an anastomosis between two structures, such as two vascular structures, using the disclosed medical devices.

### CROSS-REFERENCE(S) TO RELATED APPLICATION(S)

This application claims priority to U.S. Provisional Patent Application Ser. No. 60/458,813, filed Mar. 28, 2003, the entire contents of which are expressly incorporated by reference.

### BACKGROUND OF THE INVENTION

1. Field of the Invention

The present invention relates generally to medical devices. More particularly, it concerns medical devices for use as stents or shunts, and methods for creating an anastomosis between two structures using the same devices.

2. Description of Related Art

Numerous studies have reported the use of stents and shunts for the treatment of disease involving tubular anatomic structures (Wallace et al., 1986; Palmaz, 1988; Milroy et al., 1989; Günther et al., 1989). Placement of these devices is less invasive than surgical procedures.

Metallic stents have been widely used for treatment of vascular disease in humans. Several different types of stents have been used to treat both straight and curved vascular lesions (Gillams et al., 1990), as well as lesions occurring at vascular bifurcations (Nashef et al., 1992). Many methods for stenting lesions at a bifurcation with straight stents have been described, such as Y-stenting (Fort, 1996) and V-stent insertion (Schampaert, 1996).

Stents have also been widely used for creating connections between two vascular structures. This is typified by the use of stents to create a shunt within the liver called a transjugular intrahepatic portosystemic shunt (TIPS). TIPS has emerged as an invaluable tool in the management of morbid portal hypertension. The standard approach for TIPS, as described by Rosch et al. in 1969 in a canine model and subsequently in humans by Richter et al. in 1989, has remained relatively unchanged. A conduit is created between the right or middle hepatic vein and the right portal vein through the liver. The hepatic parenchyma acts as an in-situ covering to prevent bleeding through the interstices of the uncovered metal stent that links the two venous structures. Shunt stenosis and occlusion occur in about half of all cases after TIPS placement (Barton et al., 1995; Saxon et al., 1998). Rigorous surveillance and re-intervention to maintain respectable rates of primary assisted patency (87%) and secondary patency (89%) at 3 years (Sterling and Darcy, 1997) are required. Stenosis is believed to result from pseudointimal hyperplasia, most commonly within the outflow hepatic vein (Rossle et al., 1994).

### SUMMARY OF THE INVENTION

One embodiment is a medical device that includes a hollow body having a main portion and two ends, each end including atraumatic arms; and a graft material attached to the body; where the body is expandable from a constrained position to an unconstrained position, the atraumatic arms of one end are outwardly oriented at an acute angle with respect to the main portion when the body is in the unconstrained position, and the graft material contacts one arm that is oriented at an acute angle.

Another embodiment is a medical device that includes a self-expanding body made from shape memory material, the body having a main portion, two ends, and atraumatic arms attached to the two ends; and graft material contacting one of the atraumatic arms; where the body is expandable from a constrained position to an unconstrained position, and the atraumatic arms of both ends are outwardly oriented at an angle of no greater than 90 degrees with respect to the main portion when the body is in the unconstrained position.

Another embodiment is a method for creating an anastamosis between two structures that includes creating an opening in a first structure with an interior; creating an opening in a second structure with an interior; and positioning one of the present medical devices within the openings such that the atraumatic arms of one end of the medical device are located within the interior of the first structure and the atraumatic arms of the other end of the medical device are located within the interior of the second structure.

### BRIEF DESCRIPTION OF THE DRAWINGS

The following drawings demonstrate certain aspects of the present methods and devices. They illustrate by way of example and not limitation.

FIG. 1 is a perspective view of one of the present medical devices, showing graft attached to the inside of the body of the medical device.

FIG. 2 is a side view of one of the present medical devices, demonstrating one set of atraumatic arms oriented outwardly at an acute angle with respect to the main portion of the body, and the other set of atraumatic arms oriented outwardly at an angle of 90 degrees with respect to the main portion of the body in the unconstrained position.

FIG. 3 is a side view of one of the present medical devices, demonstrating both sets of arms oriented outwardly at an acute angle with respect to the main portion of the body in the unconstrained position.

FIG. 4 is a side view of one of the present medical devices, demonstrating graft material attached to the outside of the body of the medical device.

FIG. 5A demonstrates a flat pattern of material used to construct one of the present medical devices, with appearance of the material in a constrained position.

FIG. 5B demonstrates the material from FIG. 5A in a flat pattern following laser cutting from tubing stock.

FIG. 5C demonstrates the material from FIG. 5A in a flat pattern and an expanded position.

FIGS. 6A-6F demonstrate stages of one embodiment of placing one of the present medical devices to form an anastomosis between two vascular structures.

### DESCRIPTION OF ILLUSTRATIVE EMBODIMENTS

In this document (including the claims), the terms “comprise” (and any form of comprise, such as “comprises” and “comprising”), “have” (and any form of have, such as “has” and “having”), and “include” (and any form of include, such as “includes” and “including”) are open-ended linking verbs. Thus, a medical device “comprising” a hollow body having a main portion and two ends, each end including atraumatic arms; and a graft material attached to the body, is a medical device possessing a hollow body and a graft material attached to the body, but is not limited to only the described hollow body and attached graft material.

The terms “a” and “an” mean one or more than one. The term “another” means at least a second or more.

In some respects, the present invention provides a mechanism that can be used to create a connection between two structures with interiors, where the connection between the two structures is off-axis (meaning the axis of the device making the connection is not aligned with either of the axes of the structures being connected) and is associated with minimal obstruction of the interiors of the structures.

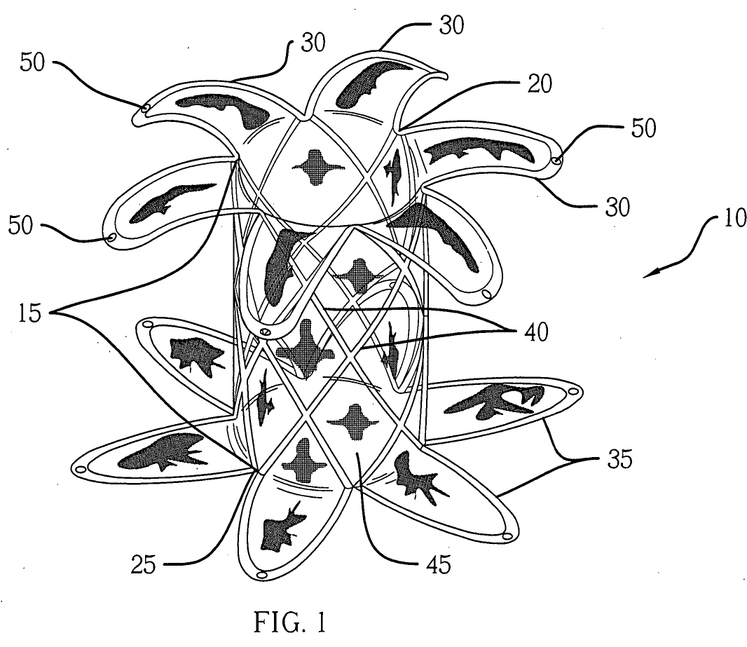

One of the present medical devices is shown in FIG. 1. Medical device 10 includes a hollow body having a main portion 15 and two ends (20 and 25). Each end includes atraumatic arms (30 and 35). The body is expandable from a constrained position to an unconstrained position. In the embodiment shown in FIG. 1, the body of the medical device is in the unconstrained position.

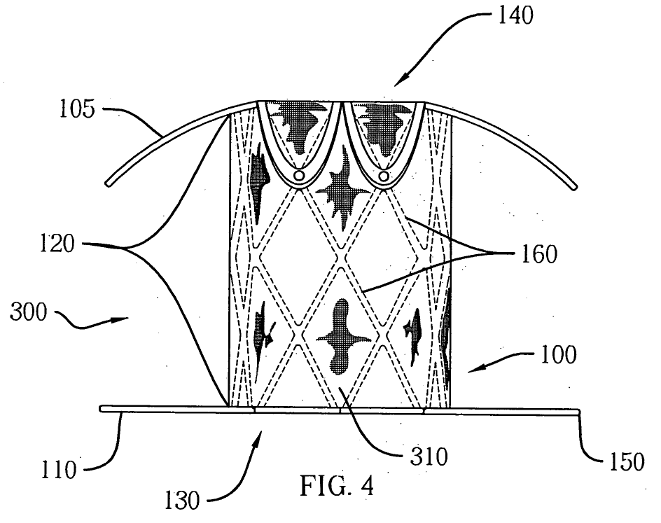

In the embodiment shown in FIG. 1, the body is composed of material having a lattice-like configuration 40. In the embodiment shown in FIG. 1, graft material 45 is attached to the inner surface of the body. In other embodiments, graft material may be attached to the outer surface of the body (see, e.g., FIG. 4), or to both the inner and outer surfaces of the body. The graft material may be attached in any suitable way known to those of skill in the art, including through the use of an adhesive, heat, a combination of the two, or by tying the graft material to the body. The body may be composed of any material capable of expanding from a constrained position to an unconstrained position. For example, the main portion can be composed of nitinol, stainless steel, or any other suitable biocompatible material known to those of skill in the art.

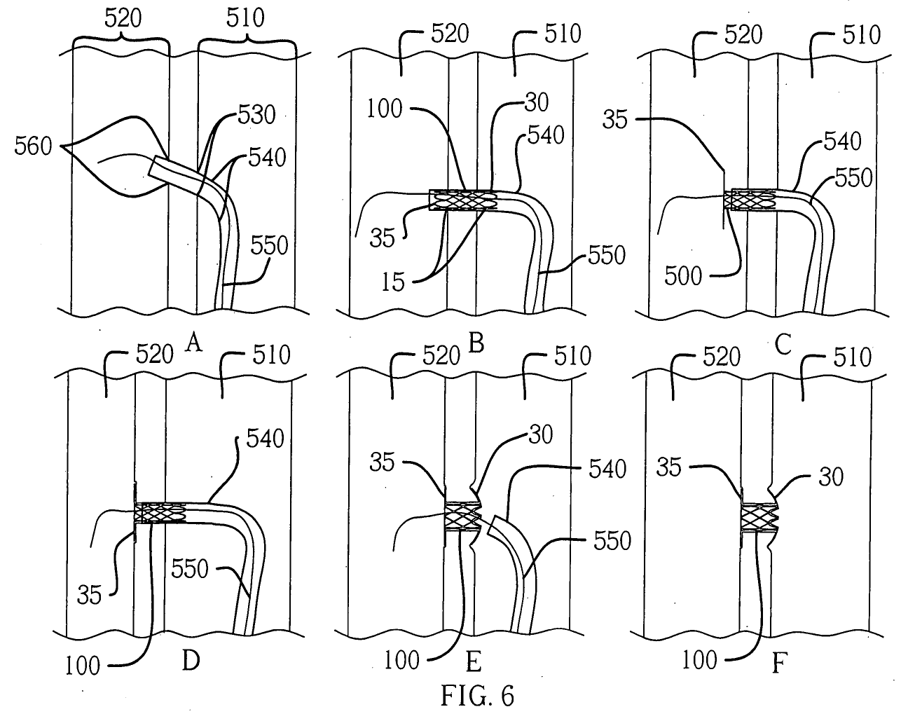

In certain embodiments, the present medical device can include a delivery device configured to deliver the body to a location within a patient. For example, the delivery device can include a catheter and a guidewire (see, e.g., FIG. 6A-6F). A constrained position can be a position of the medical device when the medical device is positioned within a catheter for delivery into a vascular structure (see, e.g., FIG. 6A). A constrained position can be a fully constrained position or a partially constrained position. An unconstrained position can include a position of the medical device between two structures following delivery, such as a position between two vascular structures (see, e.g., FIG. 6F).

Manipulation subsequent to delivery may be required to convert a constrained or partially constrained medical device to an unconstrained position. In some embodiments, a balloon may be used to convert the medical device from a constrained position to an unconstrained position. Any method known to those of skill in the art is contemplated for converting the device from a constrained to an unconstrained position. In other embodiments, the medical device is self-expanding, and no manipulation subsequent to delivery is needed for the medical device to assume the unconstrained position.

Any method known to those of skill in the art can be used to construct the body, e.g., the main portion 15 and atraumatic arms (30 and 35). In the embodiment shown in FIGS. 1-6, the body, including the atraumatic arms, may be constructed by laser cutting nitinol tubing. In other embodiments, the body, including the atraumatic arms are constructed from the same laser cut nitinol tubing but a molding form is used to bend and shape the arms. Heat is then applied while the device is in the molding form for it to retain its thermal memory configuration.

Each end of the body of the medical device shown in FIG. 1 includes atraumatic arms (30 and 35). The atraumatic arms 30 of one end 20 are outwardly oriented at an acute angle (i.e., at an angle of no greater than 90 degrees) with respect to the main portion 15 when the body is in the unconstrained position (i.e., the position shown in FIG. 1). In the embodiment shown in FIG. 1, the atraumatic arms 35 of the other end 25 are oriented at an angle of 90 degrees with respect to the main portion when the body is in the unconstrained position.

When certain of the bodies of the present medical devices are constructed through cutting tubing in some way (such as by laser cutting), it is possible to position the atraumatic arms as shown in FIG. 1 and then heat treat the body so that the arms retain that position at certain temperatures and will revert to that position when left unconstrained. Those of skill in the art having the benefit of this disclosure will understand that a mandrel, or shaping tool, may be used in such a process. For example, a body that has been cut may be placed around a mandrel, and the arms of the body may be oriented in their desired position. The arms may then be maintained in that position in any suitable way—such as by securing them to the mandrel using clamps or heat-resistant wiring—and the mandrel-body combination may be cured using heat for an appropriate period of time. Multiple mandrels may be used to facilitate curing.

Depending on the material used and the curing temperature and time chosen, certain of the bodies of the present medical devices may be cured so as to exhibit both shape memory and superelastic properties. For example, curing at about 500° Celsius for about 5 to 15 minutes should operate to impart superelastic properties to a body made from nitinol, either in lattice-structure or woven wire form. As another example, in addition to using a material such as nitinol that may impart self-expandability to the resulting medical device, the body of the medical device may be provided with a temperature dependent mechanical characteristic that allows a mechanical property of the body to be changed by heating the body from a temperature below a transition temperature to above a transition temperature. For example, where the body is made of nitinol, the body-mandrel combination may be heated to about 500° Celsius for about 60 to 120 minutes. The initial shape of the body cured in this fashion may be deformed upon application of a force at a first temperature. The force may be removed, and the body may remain deformed. The temperature dependent mechanical characteristic of the body may then be activated by heating the body to a second temperature, at which temperature the body may substantially recover its initial shape.

In the embodiment shown in FIG. 1, the atraumatic arms (30 and 35) are composed of partial loops of material. The partial loops are designed such that there would be no trauma or penetration of the tissue against which the arms rest following delivery.

The medical device may or may not include small openings in the atraumatic arms. In the embodiment shown in FIG. 1, small openings 50 are included at the tip of the arms (30 and 35) at both ends. These openings can be used to assist with loading the device into a delivery system such as a catheter (e.g., a 9-F catheter), or can be filled with a radio-opaque material (e.g., gold or platinum) to make the medical device more visible during fluoroscopy or radiographically-monitored procedures.

In the embodiment shown in FIG. 1, the medical device includes graft material 45 that is attached to the body. Graft material 45 contacts atraumatic arms 30 and 35. More specifically, graft material 45 covers (which is one manner of contacting) atraumatic arms 30 and 35. In other embodiments, the graft material may contact at least one of atraumatic arms 30 or 35. That contact may take the form of covering some or all of the arm. In the embodiment shown in FIG. 1, graft material 45 is attached to the inside of the body, including the atraumatic arms. Graft material 45 can include biocompatible material such as polytetrafluoreoethylene, polyethylene terephthalate, polyester, or any similar biocompatible material known to those of skill in the art.

The dimensions of the present medical device may be chosen based on the application for use of the device. For example, one of the present medical devices for use as a stent to create a vascular anastamosis between two vascular structures can be constructed by laser cutting a piece of nitinol tubing, where the nitinol tubing has an outer diameter of 0.203 inch (5.156 mm) and an inner diameter of 0.187 inch (4.750 mm). In the expanded position, the main portion of the body of the medical device may have a length of 0.472 inch (11.99 mm).

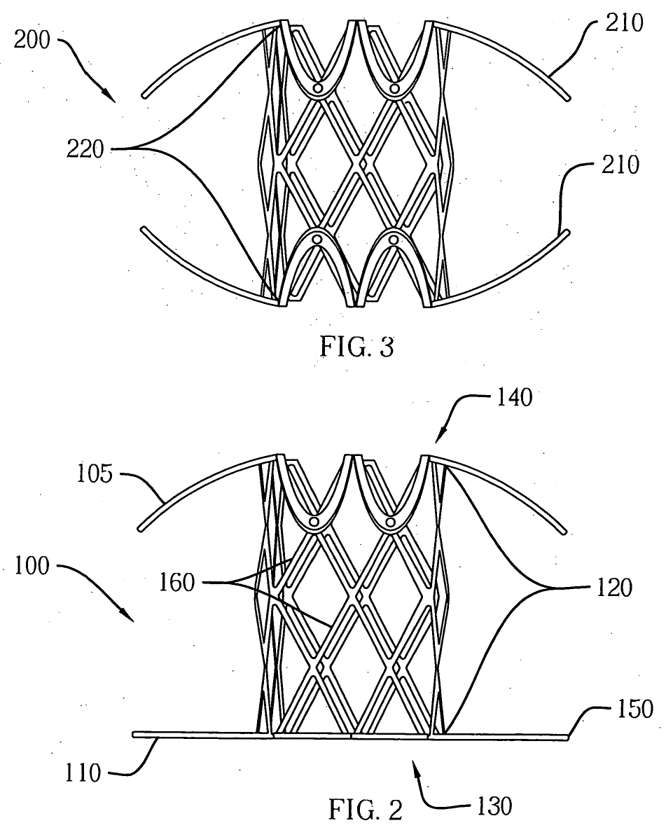

FIG. 2 demonstrates a side view of another of the present medical devices, but without graft material attached to the body. FIGS. 2 and 3 are similar in this regard, and are designed to illustrate a suitable structure for use as the body of the present medical devices. The side view of body 100 shown in FIG. 2 demonstrates a suitable orientation of atraumatic arms 105 and 110 of the ends of the body with respect to the main portion 120 when the medical device is in the unconstrained position. In the embodiment shown in FIG. 2, atraumatic arms 110 of one end 130 are outwardly oriented at an angle of 90 degrees with respect to main portion 120 when the body is in the unconstrained position. Atraumatic arms 105 of the other end 140 are oriented at an acute angle (i.e., an angle less than 90 degrees) with respect to main portion 120 when the body is in the unconstrained position. The tips 150 of the arms may either be of a smooth (e.g., rounded) configuration or a blunt (e.g., angular) configuration. Similarly, a cross section of any portion of the lattice-like frame 160 of the body 100 may demonstrate a section that has smooth edges or angular edges. As FIG. 2 shows, the positioning of the atraumatic arms of an end may be different with respect to each other than the symmetric orientation shown in FIG. 1. Furthermore, while the atraumatic arms shown in FIG. 1 are slightly arched, the atraumatic arms in FIGS. 2 and 3 are more straight.

FIG. 3 shows another embodiment of the present medical devices (without the attached graft material). The atraumatic arms 210 of the ends of body 200 are oriented at an acute angle with respect to main portion 220 when the body is in the unconstrained position (i.e., the position shown). Atraumatic arms 210 of at least one of the ends of body 200 are oriented at any angle that is less than 90 degrees with respect to the main portion 220 when body 200 is in the unconstrained position. In this embodiment, atraumatic arms 210 should not be oriented at an angle greater than 90 degrees with respect to main portion 220 when body 200 is in the unconstrained position. When body 200 is in the constrained position (see, e.g., FIG. 6B), the arms can be oriented at any angle with respect to main portion 220.

Another embodiment of the present medical devices is shown in FIG. 4. This embodiment is like the embodiment shown in FIG. 2, except it includes graft material 310 attached to the outer surface of body 100. In other embodiments, graft material may be attached to both the outer and inner surfaces of the body. Such an embodiment would look no different than what is shown in FIG. 4.

In embodiments of the present medical devices, graft material contacts at least one of the atraumatic arms. In certain of these embodiments, graft material may or may not be in contact with the body of the medical device.

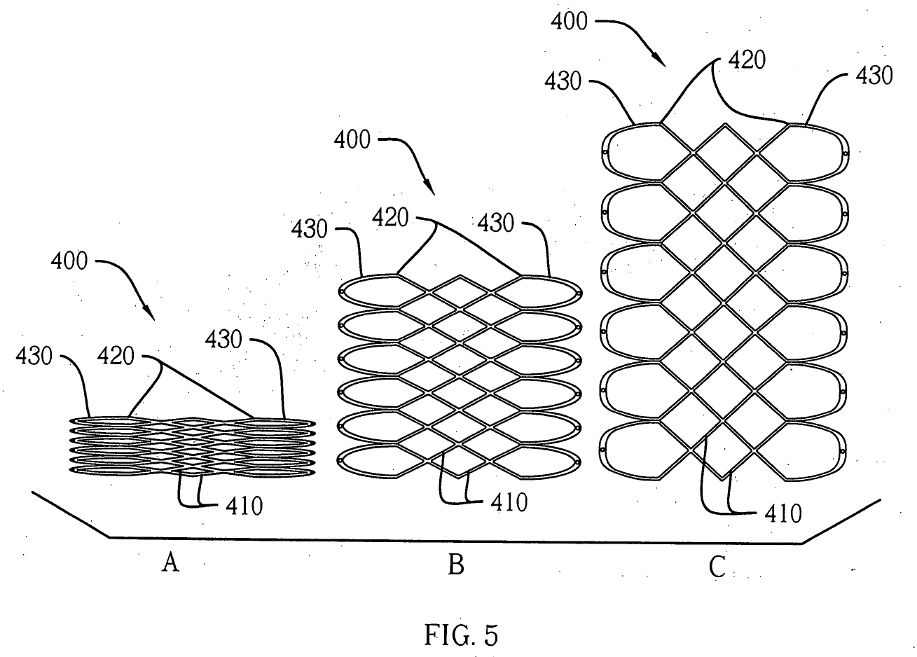

FIGS. 5A-5C demonstrate the appearance of a flat pattern of material that may be used to construct the body of one embodiment of the present medical devices. The pattern shown in the figures may be cut (e.g., by laser, chemical etching, or the like) from tube stock (e.g., nitinol tubing). FIG. 5A demonstrates the appearance of the flat pattern of body 400 in a constrained position, the body having a lattice-like structure 410, a main portion 420 and atraumatic arms 430. FIG. 5B demonstrates the appearance of the flat pattern of body 400 in an unconstrained position (e.g., as if it were placed in a flat position following cutting from tube stock). Following cutting of the tubing, the material may not be in the fully expanded position. Further manipulation of the sheet of cut tubing may be required in order for the sheet to have the final desired unconstrained position of the medical device, as shown in FIG. 5C. As previously noted, any method known to those of skill in the art can be used to construct the bodies of the present medical devices, including the atraumatic arms; cutting tubing is only one example. Any material can be used to construct the bodies of the present medical devices, as long as the bodies are capable of expanding from a constrained position to an unconstrained position. The material may be self-expanding, or may require additional manipulation to assume an unconstrained position.

FIGS. 6A-6F demonstrate stages of one embodiment of a method for using medical device 100 (see FIG. 1) for creating an anastamosis between two structures. The two structures (510 and 520) each have an interior. The two structures, for example, can be vascular structures. The vascular structures can be either veins or arteries. For example, in one embodiment, the first vascular structure is the inferior vena cava and the second vascular structure is the main portal vein. Thus, the anastamosis may, for example, be a transvenous extraphepatic portacaval shunt (TEPS). The portal vein may be tented during the practice of the method illustrated in FIGS. 6A-6F.

In the stages schematically depicted in FIG. 6A, an opening 530 in first structure 510 (in this case, a vascular structure) may be created. An opening 560 in second structure 520 may be also be created, and done in the same way. Creation of the openings in these structures can be accomplished in any suitable fashion, including using a cutting tool (such as a needle), followed by the insertion of a delivery catheter 540, or a guidewire 550. For example, the Seldinger technique may be used. In another example, the catheter may be advanced through a guiding sheath. In this regard, Angiodynamics Incorporated's (Queensbury, N.Y.) transjugular liver access set may be used. This access set contains, in relevant part, a 14-gauge blunt curved cannula (through which the other components pass; it stabilizes the system and is useful given the size of the IVC, and provides directional ability); a 67-centimeter, 21-gauge needle; and a tapered 5-F catheter that is backloaded onto the 21-gauge needle. The portal vein may be entered with the 21-gauge needle. An 0.018-inch nitinol torque guidewire with a radiopaque tip may then be threaded through the 21-gauge needle and into the portal vein. Once the portal vein is accessed and the guidewire is advanced into the lumen, the 5-F catheter may be advanced over the 21-gauge needle and into the portal vein. The IVC/portal vein tract may be scaled up, or sized up, to accept a 9-F sheath (which may be a FLEXOR sheath from Cook that is 35-centimeters long and angled, and which is represented as catheter 540 in the figures). This may be performed by placing a 0.035-inch stiff (AMPLATZ) guidewire through the 5-F catheter and into the portal vein, exchanging the 5-F catheter for the dilator that comes with the 9-F sheath, and—in a single maneuver—the 9-F dilator/sheath over the stiff guidewire into the portal vein. This is known as a single, step-up maneuver from a 5-F tract to a 9-F sheath tract.

The entry into the portal vein may be done under guidance of, for example, a 6-F, 12.5-MHz SONICATH ULTRA Boston Scientific IVUS catheter and fluoroscopy. That IVUS catheter may used to identify the portal vein, and to identify the shortest distance between the portal vein and IVC. Once the same is accomplished, the blunt curved cannula described above may directed toward the portal vein, under the guidance of the IVUS catheter, and the 21-gauge needle is advanced across the wall of the IVC; and across the portal vein wall and into the lumen of the portal vein. An 8-F, 25-centimeter PINNACLE introducer sheath from Boston Scientific/Medi-Tech may be used as the introducer sheath for the IVUS catheter.

As discussed above, ultrasound (e.g., intravascular ultrasound (IVUS)) may or may not be used to assist in creating the openings in the first and second vascular structures. Other imaging technologies, such as transabdominal ultrasonography, may alternatively be used; computed tomography and magnetic resonance imaging are other potential alternatives.

In more general detail, in the embodiment of the method that is depicted, catheter 540 may be advanced over guidewire 550 and through the openings (530 and 560) created in first and second structures 510 and 520.

FIG. 6B demonstrates positioning medical device 100 in the constrained position within catheter 540, across the openings (530 and 560) created in structures 510 and 520. Medical device 100 includes main portion 15 and two sets of atraumatic arms 35 and 30. Although not shown, medical device 100 includes a graft material attached to the inner surface of the body. After creation of the openings, the guidewire position may be maintained while the introducing catheter is withdrawn. Medical device 100 may then be constrained and placed in the same or in a different catheter. The chosen catheter, which in the embodiment shown is catheter 540, and constrained medical device 100 may then be positioned by advancing them both along guidewire 550 to a position across the two openings (530 and 560) in the first and second vascular structures 510 and 520. Ultrasound (e.g., IVUS)or fluoroscopic guidance (e.g., lateral fluoroscopic guidance) may or may not be used to assist in positioning of the medical device across the openings created in the first and second vascular structures.

FIG. 6C demonstrates the partial release of one of the present medical devices. This stage shows the expansion of one set of atraumatic arms 35 within the lumen of one of the vascular structures 520. The atraumatic arms 35 in the embodiment depicted self-expand from a constrained position where the arms are initially at an angle of roughly zero degrees with respect to the main portion of the body when the body is in the constrained position to an angle of 90 degrees with respect to the main portion of the body when the body is in the constrained position.

FIG. 6D shows the final position of atraumatic arms 35 against the interior of the wall of second vascular structure 520 following removal of catheter 540 from second vascular structure 520. Guidewire 550 is still in position within the interior of the second vascular structure. Atraumatic arms 35 are oriented at an angle of 90 degrees with respect to the main portion of the body of the medical device 100, which remains in the constrained position within the catheter. Atraumatic arms 35 are in direct contact with the interior (e.g., the wall) of second vascular structure 520.

Following removal of catheter 540, medical device 100 maintains a position across the openings created in the first 510 and second 520 vascular structures (FIG. 6E). Medical device 100 expands to an unconstrained position as shown in FIG. 6E. Guidewire 550 is maintained in position across the openings created in the first 510 and second 520 vascular structures while catheter 540 is removed. The embodiment of the medical device depicted demonstrates one set of atraumatic arms 30 oriented at an acute angle with respect to the main portion of the body of medical device 100 when the body is in the unconstrained position. Atraumatic arms 30 assume a position in direct and most-likely continuous contact with the interior of first vascular structure 510, although it is possible that not all parts of each arm 30 directly contact the interior of first vascular structure 510.

FIG. 6F demonstrates the final position of the depicted embodiment of medical device 100 following removal of the guidewire 550. When both sets of atraumatic arms 35 and 30 are in contact with the interior of the vascular structures, there is no penetration of the arms through any portion of the wall of either vascular structure (i.e., there is no traumatic disruption of the wall of either vascular structure). Final positioning of the medical device across the openings in the first and second structures may or may not result in the atraumatic arms of one end contacting the inner surface of the first structure and the atraumatic arms of the other end contacting the inner surface of the second structure. As shown in FIG. 6F, the atraumatic arms can draw the two structures closer together than they were prior to the positioning.

Those of skill in the art will appreciate that, in the detailed description above, certain well-known components and assembly techniques have been omitted so that the present medical devices and methods are not obscured in unnecessary detail. Dimensions provided in English units may be translated to the corresponding metric unit by rounding to the nearest millimeter.

All the disclosed embodiments of the invention can be made and used without undue experimentation in light of the disclosure. The individual medical devices described above need not be made in the exact disclosed forms, or combined in the exact disclosed configurations, but could be provided in any suitable form, and/or combined in any suitable configuration consistent with the claims below. Further, although the present methods can be practiced using the specific disclosed elements, such methods can also be practiced incorporating other elements or techniques consistent with the claims below. The claims are not to be interpreted as including means-plus-function limitations, unless such a limitation is explicitly recited in a given claim using the phrase(s) “means for” and/or “step for,” respectively.

### REFERENCES

The following references, to the extent that they provide exemplary procedural or other details supplementary to those set forth above, are specifically incorporated by reference.
Barton et al., Semin. Interv. Radiol., 12:364, 1995.
Fort, Can. J. Cardiol., 12(7):678-682, 1996.
Gillams et al., Radiology, 174:137-140, 1990.
Günther et al., Radiology, 170:401405, 1989.
Milroy et al., Br. J. Urol., 63:392-396, 1989.
Morita, Jap. J. Diagn. Imaging, 17(5):526-535, 1997.
Nashef et al., Ann. Thorac. Surg., 54:937-940, 1992.
Palmaz, AJR Am. J. Roetgenol, 150:1263-1269, 1988.
Richter et al., Radiologe, 29(8):406-411, 1989.
Rosch et al., Radiology, 92(5):1112-1114, 1969.
Rossle et al., N. Engl. J. Med., 330(3):165-171, 1994.
Saxon et al., Radiology, 207(3):683-693, 1998.
Schampaert, Cathet. Cardiovasc. Diagn., 39(3):320-326, 1996.
Sterling and Darcy, AJR Am. J. Roetgenol., 168(1):239-244, 1997.
Wallace et al., Radiology, 158:309-312, 1986.

1. A medical device comprising:
a hollow body having a main portion and two ends, each end including atraumatic arms; and
a graft material attached to the body;
where the body is (i) expandable from a constrained position to an unconstrained position, (ii) the atraumatic arms of one end are outwardly oriented at an acute angle with respect to the main portion when the body is in the unconstrained position, and (iii) the graft material contacts one arm that is oriented at an acute angle.

2. The medical device of claim 1, where the atraumatic arms of the other end are outwardly oriented at no greater than 90 degrees with respect to the main portion when the body is in the unconstrained position.

3. The medical device of claim 1, where the atraumatic arms of both ends are outwardly oriented at an acute angle with respect to the main portion when the body is in the unconstrained position.

4. The medical device of claim 1, where the body includes an inner surface and an outer surface, and the graft material covers the inner surface of the body, including the atraumatic arms of both ends.

5. The medical device of claim 4, where the graft material covers the inner and outer surfaces of the body, including the atraumatic arms of both ends.

6. The medical device of claim 1, where the body includes an inner surface and an outer surface, and the graft material covers the outer surface of the body, including the atraumatic arms of both ends.

7. The medical device of claim 1, where the graft material comprises polytetrafluoroethylene, polyethylene terephthalate, or polyester.

8. The medical device of claim 7, where the graft material comprises polytetrafluoroethylene.

9. The medical device of claim 1, where the body comprises a shape memory material.

10. The medical device of claim 1, where the body is self-expanding.

11. The medical device of claim 10, where the body comprises a tube into which openings have been cut.

12. The medical device of claim 1, where the atraumatic arms are loops.

13. The medical device of claim 1, further comprising a delivery device configured to deliver the body to a location within a patient.

14. A medical device comprising:
a self-expanding body made from shape memory material, the body having a main portion and two ends, each end including atraumatic arms; and
a graft material contacting one of the atraumatic arms;
where the body is expandable from a constrained position to an unconstrained position, and the atraumatic arms of both ends are outwardly oriented at an angle of no greater than 90 degrees with respect to the main portion when the body is in the unconstrained position.

15. The medical device of claim 14, where the graft material contacts all of the atraumatic arms.

16. The medical device of claim 14, where the atraumatic arms of both ends are outwardly oriented at an acute angle with respect to the main portion when the body is in the unconstrained position.

17. The medical device of claim 14, where the body comprises a tube into which openings have been cut.

18. A method for creating an anastamosis between two structures, comprising:
creating an opening in a first structure with an interior;
creating an opening in a second structure with an interior; and
positioning the medical device of claims 1 or 14 within the openings such that the atraumatic arms of one end are located within the interior of the first structure and the atraumatic arms of the other end are located within the interior of the second structure.

19. The method of claim 18, where the anastamosis is a side-to-side anastamosis between the inferior vena cava and the main portal vein.

20. The method of claim 18, where ultrasound is used during the positioning.

21. The method of claim 18, where the creation of the openings in the first and second structures includes the use of a needle and a wire.

22. The method of claim 18, where a catheter is used during the positioning.

23. The method of claim 18, where the structures each have an inner surface, and the positioning results in the atraumatic arms of one end contacting the inner surface of the first structure and the atraumatic arms of the other end contacting the inner surface of the second structure, and where the atraumatic arms draw the two structures closer together than they were prior to the positioning.
